(19) United States
(12) Patent Application Publication
(10) Pub. No.: US 2021/0267955 A1
(43) Pub. Date: Sep. 2, 2021

(54) LOW DOSE ORAL PHARMACEUTICAL COMPOSITION OF PIRFENIDONE OR SALT THEREOF

(71) Applicant: Zenvision Pharma LLP, Mumbai (IN)

(72) Inventors: Sivakumar Venkata Bobba, Navi Mumbai (IN); Bhimrao Jadhav, Navi Mumbai (IN); Dhananjay Shinde, Navi Mumbai (IN); Sunil Pophale, Navi Mumbai (IN)

(21) Appl. No.: 17/256,675

(22) PCT Filed: Jun. 25, 2019
(86) PCT No.: PCT/IB2019/055323

§ 371 Date: Dec. 29, 2020

(30) Foreign Application Priority Data
Jun. 29, 2018 (IN) 201821024204

(51) Int. Cl. A61K 31/4418; A61K 9/48; A61K 9/08; A61K 47/10; A61K 47/14

(52) U.S. Cl. CPC A61K 31/4418; A61K 9/4858; A61K 47/14; A61K 9/08; A61K 47/10; A61K 9/4866

(57) ABSTRACT

The present invention relates to Suprabioavailable oral composition of Pirfenidone or salt thereof when compared with reference product of Pirfenidone capsule [267 mg] and tablet [267 mg and 801 mg] for the treatment of idiopathic pulmonary fibrosis (IPF). The Suprabioavailable oral composition of Pirfenidone or salt thereof provides at least 5% dose reduction which minimizes adverse effects and provides more efficacy with better patient compliance in the treatment of idiopathic pulmonary fibrosis.

### FIELD OF THE INVENTION

The present invention relates to low dose oral pharmaceutical composition of Pirfenidone or salt thereof.

### BACKGROUND OF THE INVENTION

Idiopathic pulmonary fibrosis (IPF) is a type of chronic interstitial lung disease characterized by a progressive and irreversible decline in lung function. Symptoms typically include gradual onset of shortness of breath, a dry cough, loss of weight, swelling of the legs, feeling tired and nail clubbing. Complications may include pulmonary hypertension, heart failure, pneumonia, or pulmonary embolism.

The cause of IPF is unknown but certain environmental factors and exposures have been shown to increase the risk of getting IPF. Risk factors include cigarette smoking, certain viral infections, and a family history of the condition. The underlying mechanism involves scarring of the lungs. Diagnosis requires ruling out other potential causes and may be supported by a CT scan or lung biopsy.

About 5 million people are affected globally. The disease newly occurs in about 12 per 100,000 people per year. Currently, more than 80,000 adults in the United States have IPF, and more than 30,000 new cases are diagnosed each year. The disease usually affects people between the ages of 50 and 70. Males are affected more often than females. Average life expectancy following diagnosis is about four years.

A number of medications have been investigated in the past for IPF, including interferon gamma-1β, bosentan, ambrisentan and anticoagulants, N-acetylcysteine and triple therapy; but these are no longer considered effective treatment options. Many of these earlier studies were based on the hypothesis that IPF is an inflammatory disorder. Corticosteroids (prednisone), Mycophenolate mofetil, Azathioprine are not indicated for the treatment of IPF but can treat and prevent inflammation by suppressing the immune system. Several other anti-inflammatory therapies such as methotrexate, cyclophosphamide, cyclosporine, rapamycin (sirolimus), and tacrolimus have been used to treat different forms of interstitial lung disease.

Other treatments for IPF are supportive which includes oxygen therapy, pulmonary rehabilitation and vaccination. Patients may need additional oxygen, which supplied in a small portable oxygen tank, to help them breathe. Patients are also encouraged to enroll in an exercise program known as pulmonary rehabilitation to help improve their breathing. Patients should also receive immunizations for pneumonia and yearly influenza to avoid illness, which can be more severe in people with IPF. It is important to stay active, follow a healthy diet, get plenty of rest, and quit smoking. People often benefit from pulmonary rehabilitation and supplemental oxygen but oxygen therapy has not been shown to improve survival in IPF.

The damage caused by the lung scarring cannot be reversed. The goals of treatment in IPF are essentially to reduce the symptoms, stop disease progression, prevent acute exacerbations, and prolong survival. There are currently 2 medications approved for use in IPF. The Pirfenidone and Nintedanib have been shown to slow the scarring and help preserve lung function. Lung transplant surgery may be considered in some patients, most often patients who are under 70 years old who have advanced disease.

Pirfenidone belongs to the chemical class of pyridine. Chemically Pirfenidone is 5-methyl-1-phenyl-2-1(H)-pyridone or 5-methyl-1-phenyl-2-(1H)-pyridone and its molecular weight is 185.23. Its empirical formula is C₁₂H₁₁NO. Pirfenidone is represented by compound of structural formula I.

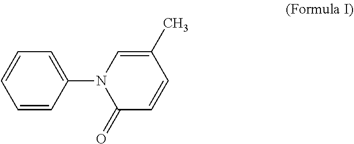

Pirfenidone is a white to pale yellow, non-hygroscopic powder. It is more soluble in methanol, ethyl alcohol, acetone and chloroform than in water and 1.0 N hydrochloric acid.

Pirfenidone capsule of Genentech Inc has been approved in USA on Oct. 15, 2014 under the trade name ESBRIET® and is available in the strength of 267 MG. The product is indicated for the treatment of idiopathic pulmonary fibrosis (IPF).

Pirfenidone tablet of Genentech Inc has been approved in USA on Jan. 11, 2017 under the trade name ESBRIET® and is available in the strength of 267 MG & 801 MG. The product is indicated for the treatment of idiopathic pulmonary fibrosis (IPF).

U.S. Pat. No. 8,519,140 discloses specifically method of synthesizing Pirfenidone. Further it generically discloses Pirfenidone may be formulated for oral administration in a lipid-based formulation suitable for low solubility compounds; however U.S. Pat. No. 8,519,140 does not discloses or teaches Suprabioavailable low dose formulation of Pirfenidone.

The product known in the prior art for Pirfenidone are available in the form of Capsule & Tablet. The product known in the prior art for Pirfenidone suffers from adverse effects such as Nausea, Rash, Abdominal Pain, Upper Respiratory Tract Infection, Diarrhea, Fatigue, Headache, Dyspepsia, Dizziness, Vomiting, Anorexia, Gastro-esophageal Reflux Disease, Sinusitis, Insomnia, Weight Decreased, Arthralgia, Agranulocytosis, Angioedema, Hepatobiliary Disorders.

Thus, there is an unmet need in the art to provide Suprabioavailable low dose composition of Pirfenidone which minimizes the adverse effects associated with Pirfenidone, provides therapeutic efficacy and patient compliance in the treatment of idiopathic pulmonary fibrosis (IPF).

### OBJECTS OF THE INVENTION

Accordingly it is an object of the present invention to provide low dose composition of Pirfenidone or salt thereof.

It is another object of the present invention to provide suprabioavailable composition of Pirfenidone or salt thereof.

It is another object of the present invention to provide suprabioavailable low dose composition of Pirfenidone or salt thereof wherein composition contain at least 5% dose reduction when compared with reference product of capsule [267 mg] & tablet [267 mg & 801 mg].

It is another object of the present invention to provide suprabioavailable low dose composition of Pirfenidone or salt thereof which provides less adverse effects.

It is another object of the present invention to provide suprabioavailable low dose composition of Pirfenidone or salt thereof in the treatment of idiopathic pulmonary fibrosis (IPF).

It is yet another object of the present invention to provide a method for treating idiopathic pulmonary fibrosis (IPF) involving administration of suprabioavailable low dose composition of Pirfenidone or salt thereof.

It is another object of the present invention to provide suprabioavailable low dose composition of Pirfenidone or salt thereof which provides better therapeutic efficacy and patient compliance in the treatment of idiopathic pulmonary fibrosis (IPF).

### SUMMARY OF THE INVENTION

A first aspect of the present invention is to provide suprabioavailable composition of Pirfenidone or salt thereof.

In another aspect of the present invention is to provide suprabioavailable composition of Pirfenidone or salt thereof; when compared with capsule [267 mg] & tablet [267 mg & 801 mg] reference product of Pirfenidone.

In another aspect of the present invention is to provide suprabioavailable low dose composition of Pirfenidone or salt thereof.

In another aspect of the present invention is to provide suprabioavailable low dose composition of Pirfenidone or salt thereof; wherein composition contain at least 5% dose reduction when compared with capsule [267 mg] & tablet [267 mg & 801 mg] reference product of Pirfenidone.

In another aspect of the present invention is to provide suprabioavailable low dose composition of Pirfenidone or salt thereof along with one or more pharmaceutically acceptable excipients.

In another aspect of the present invention is to provide process of manufacturing suprabioavailable low dose composition of Pirfenidone or salt thereof along with one or more pharmaceutically acceptable excipients.

In another aspect of the present invention is to provide low dose suprabioavailable composition of Pirfenidone or salt thereof in the treatment of idiopathic pulmonary fibrosis (IPF).

### DETAIL DESCRIPTION OF THE INVENTION

The present invention relates to Suprabioavailable composition of Pirfenidone or salt thereof.

The term Suprabioavailability according to present invention means product displays an extent of absorption appreciably larger than the reference product, capsule [267 mg] & tablet [267 mg & 801 mg] of Pirfenidone or salt thereof.

The composition according to present invention is based on the Suprabioavailability and contains at least 5% reduction of Pirfenidone dose when compared with reference product, capsule [267 mg] & tablet [267 mg & 801 mg] of Pirfenidone.

The reference products according to present invention are Pirfenidone capsule and tablet which are prepared Qualitatively similar composition as that of USA approved product of Pirfenidone i.e. ESBRIET® with the strength of 267 mg for Capsule & 267 mg and 801 mg for Tablet.

The Suprabioavailable composition of Pirfenidone or salt thereof according to present invention may be in the form of capsule i.e. hard and soft gelatin capsule, tablet, concentrated solution, granules, beads, sachet, gelcap, lozenge, pastille, pill and powder.

In another aspect of the present invention is to provide Suprabioavailable composition of Pirfenidone or salt thereof along with one or more pharmaceutically acceptable excipients.

The one or more pharmaceutically acceptable excipients according to present invention is selected from the group consisting of lipidic excipient, oily vehicle, surfactant solubilizing agent. Optionally it may contain one or more excipient selected from diluent, binder, sweetener, thickening agent, flavor, pH regulating agent, stabilizing agent, lubricant, glidant, film forming polymer, solvent, preservative, disintegrant, antiadherent and antioxidant.

The concentrations of Pirfenidone or salt thereof, excipients and the manufacturing process has been optimized in such way that it provide Suprabioavailable composition of Pirfenidone or salt thereof; when compared with capsule [267 mg] & tablet [267 mg & 801 mg] reference product of Pirfenidone. Therefore due to Suprabioavailability, it provides at least 5% reduction of Pirfenidone dose when compared with reference product capsule [267 mg] & tablet [267 mg & 801 mg] of Pirfenidone.

The Suprabioavailable composition may comprise any suitable amount of the Pirfenidone or salt thereof in order to produce an effective blood level of the Pirfenidone in the treatment of idiopathic pulmonary fibrosis (IPF). The weight percentage of Pirfenidone or salt thereof can be 5% to 60%, preferably 10% to 50% based on the total weight of composition. The amount of Pirfenidone or salt thereof according to present invention may ranges from 0.001 mg to 2000 mg, 0.001 mg to 1500 mg, 0.001 mg to 1200 mg. Further the amount of Pirfenidone or salt thereof may be equal or lesser than the reference product capsule [267 mg] & tablet [267 mg & 801 mg]. Preferably the amount of Pirfenidone or salt thereof according to present invention contains at least 5% lesser than that present in the reference formulation i.e. capsule [267 mg] & tablet [267 mg & 801 mg]; therefore amount of Pirfenidone or salt thereof may be preferably less than 254 mg or less than 761 mg.

According to present invention the lipidic excipient contributes the rapid release of the drug, good dissolution profile and ultimately achieving supra bioavailability of formulation. The examples of lipidic excipients include but not limited to glycerol macrogolglycerides, Lauroyl macrogolglycerides (Gelucire), Caprylocaproyl Polyoxyl-8 glycerides (Labrasol), polyethylene glycol derivatives, or mixtures thereof. The lipidic excipient is present in the invention in an amount of about 10% to about 90% based on the total weight of the composition; preferably in an amount of about 20% to about 80%.

According to present invention the use of an oily excipient improved the absorption of lipophilic drug by increasing the solubility of the drug in the lipidic phase. The examples of oily vehicle include but not limited to vegetable oils (Soybean oil, Peanut oil, and olive oil), medium chain triglycerides, fatty acids and their esters, fatty alcohols, amphiphilic oil, glycerol oleate derivative or mixtures thereof. An oily vehicle is present in the invention in an amount of about 5% to about 70% based on the total weight of the composition; preferably in an amount of about 10% to about 60%.

According to present invention a surfactant may be added to the composition to improve the physical stability of the formulation. The examples of surfactant include but not limited to polysorbate and its derivatives (Span 80), sodium lauryl sulphate, polyoxyethylene, sorbitan fatty acid esters, polyoxypropylene glycol or mixture thereof, derivatives of lecithin, Propylene glycol, propylene glycol esters, fatty acid esters of propylene glycol, fatty acid esters of glycerol, polyethylene glycol or mixtures thereof. The surfactant is present in the invention in an amount of about 1% to about 10% based on the total weight of the composition.

The examples of solubilizing agent include but not limited to polyethylene glycol, polyvinylpyrrolidone, dextran, triacetin, triethyl citrate, ethyl oleate, ethyl caprylate, sodium lauryl sulfate, sodium doccusate, vitamin E TPGS, dimethylacetamide, N-methyl pyrrolidone, N-hydroxy ethyl pyrrolidone, hydroxy propyl methyl cellulose, hydroxypropyl cyclodextrins, ethanol, n-butanol, isopropyl alcohol, cholesterol, bile salts, glycofurol, transcutol, propylene glycol, and dimethyl isosorbide or mixture thereof.

The examples of diluent include but not limited to mannitol, sorbitol, xylitol, cellulose derivatives, starch, maltodextrin, dibasic calcium phosphate, tribasic calcium phosphate, calcium sulphate, microcrystalline cellulose, dextrose, fructose, sucrose, lactitol, lactose, magnesium carbonate, magnesium oxide, kaolin, precipitated calcium carbonate, or mixtures thereof.

The examples of binder include but not limited to, ethyl cellulose, gelatin, hydroxyethyl cellulose, carboxymethyl cellulose sodium, hydroxymethyl cellulose, hydroxypropyl cellulose, hypromellose, magnesium aluminium silicate, maltodextrin, methyl cellulose, povidone, starch, polyvinylpyrrolidone, acacia, alginic acid, compressible sugar, liquid glucose, or mixtures thereof.

The examples of sweetener include but not limited to saccharose, glucose, a fluorinated sucrose derivative, dextrose, maltose, galactose, and artificial sweeteners, such as acesulfame potassium, sodium saccharin, saccharin and aspartame cyclamates, sucralose or mixtures thereof.

The examples of thickening agent include but not limited to guar gum, gelatin, locust bean gum, tara gum, xanthan gum, tamarind gum, tragacanth gum, karaya gum, konjac mannan, water-soluble carboxyvinyl polymer, sodium carboxymethylcellulose, sodium alginate, pectin, Azotobacter vinelandii gum, carrageenan, polyethylene glycol, modified starch, cassia gum, psyllium seed gum, carboxymethylcellulose, hydroxypropyl cellulose, hydroxypropyl methylcellulose, hydroxyethyl cellulose, methyl cellulose and microcrystalline cellulose.

The examples of flavor include but not limited to fruit flavor, peppermint flavor or mixture thereof.

The examples of pH regulating agent include but not limited to fumarate, citrate, phosphate, carbonate, tartrate, acetate, amino acid salts, sodium hydroxide, potassium hydroxide, sodium dihydrogen phosphate, disodium hydrogen phosphate, sodium carbonate, sodium bicarbonate, potassium hydrogen phosphate, dipotassium hydrogen phosphate, tripotassium phosphate, buffers like sodium citrate and citric acid or mixture thereof.

The examples of stabilizing agent include but not limited to tocopherol, cyclodextrin, tetrasodium edetate, nicotinamide, aminopolycarboxylic acids and salts thereof, iminodiacetic acid, methyliminodiacetic acid, nitrilotriacetic acid, ethylenediaminetetraacetic acid (EDTA), diethylenetriaminepentaacetic acid, 1,2-diaminocyclohexane-tetraacetic acid, N-hydroxyethylenediaminetriacetic acid or mixture thereof.

The examples of lubricant include but not limited to calcium stearate, magnesium stearate, sodium stearate, polyethylene glycol, sodium benzoate, potassium benzoate, sodium lauryl sulphate, magnesium lauryl sulphate, talc, stearic acid, sterotex (hydrogenated soybean oil), waxes, glyceryl behenate, liquid paraffin, zinc stearate or mixture thereof, boric acid, sodium benzoate, sodium oleate, sodium acetate, sodium stearyl fumarate, polyethylene glycol, hydrogenated castor oil, silica, colloidal silica, cornstarch, calcium silicate, magnesium silicate, silicon hydrogel or mixture thereof.

The examples of glidant include but not limited to colloidal silica, pyrogenic silica, hydrated sodium silicoaluminate, magnesium stearate, fumed silica (colloidal silicon dioxide), starch, talc, calcium silicate, magnesium silicate, silicon hydrogel or mixture thereof.

The examples of film forming polymer include but not limited to ethylcellulose, hydroxypropyl methylcellulose, hydroxypropylcellulose, polyvinyl alcohol, polyvinyl acetate methylcellulose, carboxymethyl cellulose, hydroxymethylcellulose, hydroxyethylcellulose, cellulose acetate, hydroxypropyl methylcellulose phthalate, cellulose acetate phthalate, cellulose acetate trimellitate; waxes; methacrylic acid polymers.

The examples of solvent include but not limited to methanol, acetone, methylene chloride, propylene glycol, polyethylene glycol, sorbitol and glycerin and purified water.

The examples of preservative include but not limited to benzoic acid, sodium benzoate, and various combinations of methyl-, propyl-, and butyl parabens.

The examples of disintegrant include but not limited to croscarmellose sodium, carboxymethyl cellulose, chitosan, carboxymethyl cellulose calcium, carboxymethyl cellulose sodium, crospovidone, hydroxypropyl cellulose, microcrystalline cellulose, methyl cellulose, starch, povidone and its derivatives, sodium starch glycolate or mixture thereof.

The examples of antiadherent include but not limited to talc, corn starch, colloidal silica, DL-leucine, sodium lauryl sulphate, stearates or mixture thereof.

The examples of antioxidant include but not limited to sodium metabisulfite, tocopherols such as alpha tocopherol, sodium bisulfate, sodium sulfite, thioglycerol, butylated hydroxyanisole, butylated hydroxytoluene, propyl gallate, acetyl cysteine, ascorbic acid, sodium ascorbate, potassium or sodium salts of sulphurous acid or mixtures thereof.

In another aspect of the present invention is to provide process of manufacturing suprabioavailable composition of Pirfenidone or salt thereof along with one or more pharmaceutically acceptable excipients.

The Suprabioavailable composition of Pirfenidone or salt thereof along with one or more pharmaceutically acceptable excipients may be manufactured by the techniques known in the art for capsule i.e. hard and soft gelatin capsule, tablet, concentrated solutions, granules, beads, sachet, gel cap, lozenge, pastille, pill and powder.

The Suprabioavailable composition of Pirfenidone or salt thereof along with one or more pharmaceutically acceptable excipients may be manufactured by process such as direct compression, dry granulation, wet granulation, roller compaction, hot melt extrusion, encapsulation, hand (manually) filling, machine filling, plate process, rotary die process and reciprocating die process.

The Suprabioavailable composition of Pirfenidone or salt thereof according to present invention is preferably manufactured by process which involves mixing Pirfenidone with one or more of the pre-melted lipidic excipients and/or oily vehicle/solvents; Further addition of surfactant optionally, along with one or more pharmaceutically acceptable excipients to form a homogenous mixture. The said homogenous mixture were converted in to the suitable dosage form like capsule i.e. hard and soft gelatin capsule, tablet, concentrated solution, granules, beads, sachet, gelcap, lozenge, pastille, pill and powder.

The Suprabioavailable composition of Pirfenidone or salt thereof according to present invention provides more oral bioavailability than capsule and tablet of reference product. The primary mechanism of action by which a lipid composition according to present invention leads to improved bioavailability is usually avoidance of the slow dissolution process. Preferably the composition allows the drug to remain in a dissolved state throughout its transit in the GIT. The drug for absorption can be enhanced by formulation of the drug as a solubilizate within a colloidal dispersion which in turn enhanced oral bioavailability enabling reduction in dose, more consistent temporal profiles of drug absorption, selective targeting of drug toward specific absorption window in GIT, and protection of drug from the hostile environment in gut. Use of Lipidic excipient and oily vehicle allows improving the absorption of Pirfenidone by increasing the solubility of the drug in the lipidic phase, a maximum bioavailability is achieved by preparing and keeping the drug in the amorphous/solubilized state in a lipid-based formulation. Additional advantage of adding surfactant improved the physical stability of the formulation.

The Suprabioavailable low dose composition of Pirfenidone or salt thereof according to present invention provides more efficacy and better patient compliance in the treatment of idiopathic pulmonary fibrosis with minimum side effects such as Nausea, Rash, Abdominal Pain, Upper Respiratory Tract Infection, Diarrhea, Fatigue, Headache, Dyspepsia, Dizziness, Vomiting, Anorexia, Gastro-esophageal Reflux Disease, Sinusitis, Insomnia, Weight Decreased, Arthralgia, Agranulocytosis, Angioedema, Hepatobiliary Disorders.

The Suprabioavailable composition of the Pirfenidone or salt thereof according to present invention were evaluated for parameters like appearance, intestinal absorption study, average weight, assay, release profile and related substance found to be in the compliance.

The Intestinal Absorption study was performed to demonstrate the Suprabioavailability of test product according to present invention in comparison with that of reference product.

The test products and reference products of the present invention were compared for intestinal absorption using In vitro Intestinal Absorption study. The test product of Example 1 (a) was compared with Example 1(b) reference product. It was found that the intestinal absorption was 5.34% more than that the reference product. The test products of Example 2 (a) and 2 (b) were compared with Example 2(c) reference product. It was found that the intestinal absorption was 33.82% and 58.50% respectively more than that of the reference product. The results of study showed that absorption of test products were appreciably larger than the reference products. Further it was observed that absorption of test products was at least 5% greater than that of reference products. Therefore test products were found to be Suprabioavailable in comparison with that of reference product. The Suprabioavailability according to present invention allows reformulating reference product to at least 5% lower dosage pharmaceutical composition compared to reference product.

The Intestinal Absorption study was performed in the following way:

### Materials and Methods

### Materials:

1. Part of a large intestine of freshly sacrificed goat.
2. Test Product.
3. Reference product (Qualitatively similar composition as that of ESBRIET®).

### Equipment/Instrument/Utensils:

USP Type II Dissolution Apparatus.
Glassware.
5.0 ml Syringes and Needles.

### Procedure:

1. Intestine of freshly sacrificed goat was thoroughly washed with running tap water to remove any contents of intestine.
2. Pieces of 7-8 cm length were cut using sharp blade and washed again.
3. Drug product was dispersed in water to obtain 0.5 PPM concentration.
4. One end of intestinal piece was tied tightly with a thread and 5 mL of 0.5 PPM solution or dispersion of drug product was added from open end which was then closed using thread.
5. Solution filled piece of intestine was then tied to the paddle of USP type II dissolution.
6. Dissolution study was carried out at 20 RPM for 1 hr.
7. Approximately 2-3 ml samples were collected from intestinal sac using needle fitted to syringe.
8. Samples were filtered through 0.22 micron membrane and analysed by HPLC to estimate unabsorbed drug. Absorbed drug was calculated by subtracting unabsorbed drug from initial sample.

The Suprabioavailable composition of Pirfenidone or salt thereof according to present invention may be packaged into the suitable container like bottles (glass and plastic), blister, strip, pouch or sachet, air tight, tamper resistant, light resistant containers and moisture proof packs and optionally oxygen busters or any packaging material suitable.

### EXAMPLES

The following Examples are provided solely for illustrative purposes and are not meant to limit the invention in any way.

### Example 1 (a): Pirfenidone Capsule (267 mg) (Test Product)

| Sr. No. | Ingredients | mg/unit | % w/w |
|---|---|---|---|
| 1. | Pirfenidone | 267 | 33.37 |
| 2. | Labrasol (Caprylocaproyl | 533 | 66.63 |
| | Polyoxyl-8 glycerides) | | |

| | | |
|---|---|---|
| Total weight | 800 | 100.00 |

Manufacturing Process:
1. Labrasol was heated in glass beaker at 40-45° C.
2. Pirfenidone was dispersed in above phase with continuous stirring.
3. Warm homogenous mixture was filled in size “2” capsules.

### Example 1 (b): Pirfenidone Capsule (267 mg) (Reference Product)

| Sr. No. | Ingredients | mg/unit | % w/w |
|---|---|---|---|
| 1. | Pirfenidone | 267 | 66.75 |
| 2. | Microcrystalline cellulose (MCC) | 113 | 28.25 |
| 3. | Povidone | 08 | 2.00 |
| 4. | Croscarmellose sodium | 08 | 2.00 |
| 5. | Magnesium stearate | 04 | 1.00 |
| 6. | Size “00” Capsules | — | — |

| | | |
|---|---|---|
| Total weight | 400.0 | 100.00 |

Manufacturing Process:
1. Pirfenidone, MCC, Povidone and Croscarmellose sodium were co-sifted through #40 sieve and blended in polybag for 10 min
2. Magnesium stearate was passed through #60 sieve and added to blend of step 1 and mixed for 3 minutes.
3. Lubricated blend from step 3 was filled in a size “00” capsules shells using suitable machine.

### Example 2 (a): Pirfenidone Concentrated Solution 801 mg (Test Product)

| Sr. No. | Ingredients | mg/unit | % w/w |
|---|---|---|---|
| 1. | Pirfenidone | 801 | 17.23 |
| 2. | Propylene Glycol | 900 | 19.35 |
| 3. | Polyethylene Glycol 400 | 2949 | 63.42 |

| | | |
|---|---|---|
| Total weight | 4650 | 100.00 |

Manufacturing Process:
1. Propylene glycol and Polyethylene Glycol 400 were mixed using stirrer at room temperature.
2. Above mixture was heated to 60-70° C.
3. Mixture was allowed to cool to 40-45° C.
4. Pirfenidone was dispersed in above phase with continuous stirring.
5. Mixing was continued till clear solution was achieved.

### Example 2 (b): Pirfenidone Concentrated Solution 801 mg (Test Product)

| Sr. No. | Ingredients | mg/unit | % w/w |
|---|---|---|---|
| 1. | Pirfenidone | 801 | 17.23 |
| 2. | Propylene Glycol | 900 | 19.35 |
| 3. | Labrasol (Caprylocaproyl | 449 | 9.66 |
| | Polyoxyl-8 glycerides) | | |
| 4. | Polyethylene Glycol 400 | 2500 | 53.76 |

| | | |
|---|---|---|
| Total weight | 4650 | 100.00 |

Manufacturing Process:
1. Propylene glycol, Polyethylene Glycol 400 and Labrasol were mixed using stirrer at room temperature.
2. Above mixture was heated to 60-70° C.
3. Mixture was allowed to cool to 40-45° C.
4. Pirfenidone was dispersed in above phase with continuous stirring.
5. Mixing was continued till clear solution was achieved.

### Example 2 (c): Pirfenidone Tablet 801 mg (Reference Product)

| Sr. No. | Ingredients | mg/unit | % w/w |
|---|---|---|---|
| 1. | Pirfenidone | 801 | 77.77 |
| 2. | Microcrystalline cellulose (MCC) | 139 | 13.47 |
| 3. | Povidone | 20 | 1.95 |
| 4. | Croscarmellose sodium | 20 | 1.95 |
| 5. | Colloidal silicon dioxide | 10 | 0.97 |
| 6. | Magnesium stearate | 10 | 0.97 |
| 7. | Titanium dioxide | 10 | 0.97 |
| 8. | Macrogol | 5 | 0.49 |
| 9. | Talc | 10 | 0.97 |
| 10. | Iron oxide | 5 | 0.49 |

| | | |
|---|---|---|
| Total weight | 1030.0 | 100.00 |

Manufacturing Process:
1. Pirfenidone, MCC, Povidone and Croscarmellose sodium were co-sifted through #40 sieve and blended in polybag for 10 min
2. Colloidal silicon dioxide was passed through #40 sieve and added to the blend of step 1 and blended for 5 minutes.
3. Magnesium stearate was passed through #60 sieve and added to blend of step 2 and mixed for 3 minutes.
4. Lubricated blend from step 3 was compressed using suitable tooling
5. Compressed tablets from step 4 were coated using coating solution.

1. A Suprabioavailable pharmaceutical composition comprising Pirfenidone or salt thereof; when compared with reference product capsule of 267 mg & tablet of 267 mg and 801 mg.

2. The pharmaceutical composition according to claim 1; wherein composition contains equal or low dose of Pirfenidone or salt thereof when compared with reference product capsule of 267 mg & tablet of 267 mg and 801 mg.

3. The pharmaceutical composition according to claim 2; wherein composition contains 267 mg or 801 mg of Pirfenidone or salt thereof.

4. The pharmaceutical composition according to claim 2; wherein composition contains less than 267 mg or less than 801 mg of Pirfenidone or salt thereof.

5. The pharmaceutical composition according to claim 2; wherein composition contains at least 5% dose reduction Pirfenidone or salt thereof, when compared with reference product capsule of 267 mg & tablet of 267 mg and 801 mg.

6. The pharmaceutical composition according to claim 5; wherein dose of Pirfenidone or salt thereof is less than 254 mg.

7. The pharmaceutical composition according to claim 5; wherein dose of Pirfenidone or salt thereof is less than 761 mg.

8. The pharmaceutical composition according to claim 1 comprises one or more excipients selected from lipidic excipient, oily vehicle and surfactant.

9. The pharmaceutical composition according to claim 8, wherein lipidic excipient is selected from the group consisting of glycerol macrogolglycerides, Lauroyl macrogolglycerides, polyethylene glycol derivatives, Caprylocaproyl Polyoxyl-8 glycerides or mixtures thereof.

10. A pharmaceutical composition according to claim 8, wherein oily vehicles selected from the group consisting of vegetable oils, medium chain triglycerides, fatty acid esters, amphiphilic oil, glycerol oleate derivative or mixtures thereof.

11. The pharmaceutical composition according to claim 8, wherein surfactant is selected from the group consisting of polysorbate and its derivatives (Span 80), sodium lauryl sulphate, polyoxyethylene, sorbitan fatty acid esters, polyoxypropylene glycol or mixture thereof, derivatives of lecithin, Propylene glycol, propylene glycol esters, fatty acid esters of propylene glycol, fatty acid esters of glycerol, polyethylene glycol or mixtures thereof.

12. The pharmaceutical composition according to claim 8, wherein lipidic excipients are in the range of 20% to 80%, oily vehicles are in the range of 10% to 60% and surfactants are in the range of 1% to 10%.

13. The pharmaceutical composition according to claim 1; wherein the composition is in the form of capsule i.e. hard and soft gelatin capsule, tablet, concentrated solution, granules, beads, sachet, gelcap, lozenge, pastille, pill and powder.

14. The pharmaceutical composition according to claim 1; wherein the composition is used for the treatment of idiopathic pulmonary fibrosis (IPF).

15. The pharmaceutical composition according to claim 4; wherein composition contains at least 5% dose reduction Pirfenidone or salt thereof, when compared with reference product capsule of 267 mg & tablet of 267 mg and 801 mg.
